(19) United States
(12) Patent Application Publication
(10) Pub. No.: US 2008/0299187 A1
(43) Pub. Date: Dec. 4, 2008

(54) Substances for Reducing Occurence of Major Cardiac Events in Humans

(76) Inventors: Joar Opheim, Aptos, CA (US); Douglas MacKay, Madbury, NH (US)

Correspondence Address: FOOTHILL LAW GROUP, 777 N. FIRST STREET, SUITE 325, SAN JOSE, CA, 95112, US

(21) Appl. No.: 11/757,340

(22) Filed: Jun. 1, 2007

(51) Int. Cl. A61K 36/06; A61K 31/20; A61K 31/341; A61K 31/351; A61K 9/48; A61P 9/10; A61K 36/53; A61K 31/355; A61K 35/60

(52) U.S. Cl. 424/452; 424/195.16; 424/523; 424/745; 514/458; 514/460; 514/474; 514/560

(57) ABSTRACT

A medicament comprising a dispersion of Red Yeast Rice extract in Omega-3 Oils. The medicament is supplied in capsules such that a daily dose is dispensed in an integral number of capsules. A dispersant is used, preferably Lysine and bamboo. The ratio of Red Yeast Rice Extract to EPA+DHA is in the range between about 1.4 and 2.8. The medicament reduces cholesterol, triglycerides, and reduces serious heart incidents.

### BACKGROUND OF THE INVENTION

1. Field of the Invention

The invention relates to compositions and methods of use of supplements and medicaments, which combine Red Yeast Rice extract, having hydroxymethylglutaryl-CoA (HMG-CoA) reductase inhibitor activity, with omega-3 fatty acid components derived from fish oils in a combined unit dose.

2. Description of the Prior Art

The statins (which are members of a group of HMG-CoA reductase inhibitors) are a group of hypolipidemic agents, used as pharmaceutical agents to lower cholesterol levels in people with or at risk for cardiovascular disease. They lower cholesterol by inhibiting the enzyme HMG-CoA reductase, which is the rate-limiting enzyme of the mevalonate pathway of cholesterol synthesis. Inhibition of this enzyme in the liver stimulates low-density lipoprotein (LDL) receptors, resulting in an increased clearance of LDL, so-called “bad cholesterol”, from the bloodstream and a decrease in blood cholesterol levels.

Statins, are potent cholesterol-lowering agents, and have been reported to lower LDL cholesterol by 30-50%.¹ Statins are classified as either synthetic or fermentation derived. Lovastatin was isolated from a strain of Aspergillus terreus and it was the first statin approved by the FDA as a drug (August 1987). Lovastatin is a water insoluble, white crystalline solid. The aqueous insolubility of lovastatin leads to inadequate dissolution in gastrointestinal fluids and, hence, poor absorption, distribution, and targeted organ delivery. Solubility of lovastatin is enhanced by reaction with β-cyclodextrin an oligosaccharide which improves the solubility of lovastatin. The improvement of aqueous solubility in such a case is a valuable goal to improve therapeutic efficacy. Lovastatin can also produce slight to moderate increases in high density lipoproteins (HDL) (10-20%), and slight decreases in triglycerides (5-10%). The usual daily dose of lovastatin is 20-80 mg/day. The statin drugs include lovastatin, pravastatin, fluvastatin, atorvastatin, simvastatin, rosuvastatin, and cerivastatin. ¹ Jones P. Kafonek S, Laurora I, et al_“Comparative dose efficacy study of atorvastatin versus simvastatin, pravastatin, lovastatin, and fluvastatin in patients with hypercholesterolemia (the CURVES study)” Am J Cardiol 1998; 81-(5): 582-7.

Compounds similar to lovastatin have also been found as an ingredient in a natural fermentation product known as Red Yeast Rice, which are also members of the HMG-CoA inhibitors. A monograph published in Alternative Medicine Review (Volume 9, Number 2, 2004) reports that the HMG-CoA reductase inhibitor activity in Red Yeast Rice comes from a naturally occurring family of nine compounds called monacolins, each of which has HMG-CoA reductase inhibitor activity. Additional active ingredients in Red Yeast Rice include sterols (beta-sitosterol, campesterol, sigmasterol, and sapogenin), isoflavones, and monounsaturated fatty acids². One of the monacolins, monacolin K, is said to be the lactone form of the statin drug lovastatin, which is converted to the active acid form in vivo by the liver. Red yeast rice is a common foodstuff in Asian countries where the average daily intake is 14-55 grams. The nutritional supplement derived from Red Yeast Rice is Red Yeast Rice extract, which is obtained by drying the fermented product of rice on which the yeast Monascus purpureus has been grown and extracting the dried product with a solvent, usually aqueous ethanol or water. The Red Yeast Rice extract contains about 0.2% monacolin K and about 0.5% total monacolins. ² Heber D. et al. Cholesterol Lowering effects of Proprietary Chinese red yeast rice dietairy supplement, Am J Clin Nutr 19999; 69:231-236.

U.S. Pat. No. 6,046,022 to Zhang, et al (Peking University), discloses some methods of making high lovastatin (monacolin K) red yeast rice and using red yeast rice and red yeast rice extract. U.S. Pat. No. 6,046,022 is hereby incorporated by reference herein in its entirety.

U.S. Pat. Nos. 6,541,005, 6,436,406, 6,495,173, 6,544,525, 6,576,242, 6,541,006 and 6,410,521 all issued to Baswell disclose methods of using red yeast rice, and all of these patents are incorporated herein by reference in their entirety.

U.S. Application 20060211763 discloses a statin drug dissolved in [a]solvent system comprising natural or synthetic omega-3 fatty acids and U.S. Application 20060034815 discloses novel omega-3 oil solutions of one or more statins.

Omega-3 fatty acids are primarily derived from fish oils and are known to reduce serum triglycerides³ and adverse coronary events. The principal active ingredients in fish oil are eicosapentaenoic acid (EPA) and docosahexaenoic acid (DHA), which were given at a combined dose of 4 g/day for seven months to hypertriglyceridemic patients resulting in a reduction of 47% in triglycerides⁴. ³ Abe Y. El-Masri B. et al. Soluble cell adhesion molecules in hypertriglyceridemia and potential significance on monocyte adhesion. Arteriosler Thromb Vasc Biology 1998:18:723-731.⁴ Ridker, Paul, Effects of n-3 Fatty Acid Therapy on Lipids and sCAMs—Inflammatory Markers. Pharmacotherapy and Clinical Trials, Lipids Online.org, posted: Oct. 3, 2001, reviewed Oct. 4. 2001.

The effects of statin drugs and omega-3 are cumulative. When 59 patients who were already receiving 10-40 mg daily of the statin simvastin were given 2 grams twice daily of EPA+DHA, there was a further sustained significant decrease of 20-30% in triglycrides.⁵ ⁵ Durington P. Bhatnager, et al. An omega-3 polyunsaturated fatty acid concentration administered for one year decreased triglycerides in simvastin treated patients with CHD. Heart 2001:85(5) 544-548.

Omega-3s are also well known to those skilled in the art to reduce inflammation, decrease arrhythmias, decrease risk of sudden cardiac death and cardiac arrest.

Considering the similarities and advantages of Red Yeast Rice compared to the statin drugs, and the complementary nature of statin drugs and Omega-3 fatty acids, there is a need for a combined Red Yeast Rice Extract with Omega-3 fatty acids in a combined unit dose form

### SUMMARY OF THE INVENTION

The invention comprises red yeast rice extract dispersed in omega-3 fatty acids. The red yeast rice extract preferably comprises, at least one and preferably more than one monacolin compounds, including monacolin K preferably at least one other monacolin and more preferably all of the other nine monacolins. The red yeast rice extract preferably contains about 0.2% monacolin K and 0.5% of total monacolins. The red yeast rice is the extract of fermenting-white rice, preferably non-glutinous white rice, with Monascus purpureus strain of yeast, culturing said Monascus purpureus strain in a culture medium comprising rice at a temperature of about 15° C. to about 35° C. for a period of about 2 to about 20 days to provide a crude fermentation product containing red rice; drying said crude fermentation product to obtain red yeast rice, and extracting said red yeast rice with a solvent to provide an extract; and-drying said extract to remove the solvent and produce red yeast rice extract. The solvent is preferably either aqueous ethanol or water. Other culture media may be added to the rice. For example, sugar, an additional carbon source chosen from the group consisting of glycerine, malt, and potato juice; thick beefjuice, and defoamer. The red yeast rice extract preferably contains about 0.2% monacolin K and 0.5% of total monacolins. Red Yeast Rice extracts are readily available in commerce in the United States and may be purchased already prepared. The preferred daily dose of red yeast rice extract is 1.2 to 2.4 grams per day, which were the dosages used in human clinical trials.

It is preferred to use a high quality source of omega-3, which is rich in Omega-3 Oils, preferably at least 70% Omega-3 Oils, and also rich in EPA and DHA. Preferably, at least-. 75% of the Omega Oils are EPA+DHA, and more preferably 85% or more are EPA+DHA. The daily dose of Omega-3 oils is 1-4 grams of Omega-3. One possible source is a balanced Omega-3 formula such as Nordic Naturals, Inc. ProOmega, which is 70% Omega-3 oils of which 50.8% is EPA, 35.1% is DHA and 14.1% is other Omega-3. Another preferred omega-3 source is Pro-EPA, which comprises 69.1% EPA, 16.3% DHA, and 14.6% other omega-3. Still another preferred source is Nordic Naturals Pro-EFA xtra which comprises 56.9% EPA, 14.7% DHA, 17.2% GLA, and 11.2% other Omega-3. The Pro-EFA xtra formula adds an Omega-6, GLA, and makes a powerful anti-inflammatory mixture.

The Red Yeast Rice extract is water soluble and is not soluble in the Omega-3 Oil. A dispersant is preferably used to keep the Red Yeast Rice extract in suspension. One preferred dispersant is 70% silica bamboo with Lysine made from Sunflower oil. A suitable mixture is to vigorously mix 24 weights of Red Yeast Rice Extract with 36 weights of Fish Oil, 1.2 weights of Bamboo (2%), and 1.8 weights of Lysine (3%). The mixture may then diluted to the desired omega oil to red yeast rice extract ratio. The omega-3/red yeast rice extract is then preferably encapsulated in soft gelatin capsules for dispensing. Preferably the capsules are of such a size that an integral number of capsules comprise a daily dosage of the mixture.

A dosage of the mixture further preferably includes antioxidants such as Rosemary, vitamin E, astaxanthine, carnitine, ascorbyl palmitate, tocopherols or other antioxidants known in the art for stabilizing fish oil.

It is an OBJECT of the invention to provide a medicament comprising a combined dispersion of Red Yeast Rice Extract in high quality Omega-3 Fatty Acids in unit dose form to make available a medicament for reducing blood lipids and preventing harmful coronary events.

### BRIEF DESCRIPTION OF THE DRAWINGS

There are no drawings with this application, containing only composition and process claims.

### DESCRIPTION OF THE PREFERRED EMBODIMENTS

Comparison between Red Yeast Rice Extract and Lovastatin Red Yeast Rice Extract contains monacolin K, the lactone form of the statin drug Mevacor® (lovastatin). Red Yeast Rice extract has been tested in clinical trials at daily dosages of 1.2 g and 2.4 g. The monacolin K content of the Red Yeast Rice Extract used in the clinical trials was 0.20% of the Red Yeast Rice extract. The monacolin K dose was therefore 2.4 to 4.8 mg/day. At 2.4 mg/day of monacolin K, the total cholesterol, LDL cholesterol, and triglycerides dropped by 23%, 31%, and 34% respectively. At 4.8 mg/day, the reduction was 17%, 23%, and 16% respectively.⁶ ⁶ Monograph by Thorne Research Inc., Alternative Medicine Review, Volume 9. Number 2, 2004.

Lovastatin has been shown to have a cholesterol lowering effect in doses ranging from 10 to 80 mg/day⁷. A study was performed to show the effectiveness of low-dose lovastatin in lowering serum cholesterol⁵. Fifty-six patients were given 20 mg/day of lovastatin for 24 weeks. Total cholesterol fell by 26% and triglycerides fell by 12%. ⁷ Bates, M, et al. Effectiveness of low dosage lovastatin in lowering serum cholesterol. Experience with 56 patients. Archives of Internal Medicine 1990:150(9)

Mevacor® (lovastin) in its package insert reported extensive clinical trials at dosages of 10, 20, and 40 mg/day. Total cholesterol was reduced in the range from 16-24%, LDL was reduced by 21-32%, and triglycerides were reduced by 10 to 6% (higher reduction observed at lower dosage).

It should be noted that Red Yeast Rice at a dosage of 2.4 mg/day of monacolin K produced better lipid reducing results than Mevacor at 10-40 mg/day. It is therefore unlikely that the lipid lowering effects with Red Yeast Rice result from the monacolin K content alone of Red Yeast Rice, but are probably attributable in whole or in part to the other 8 monacolins and sterols (beta-sitosterol, campesterol, sigmasterol, and sapogenin), isoflavones, and monounsaturated fatty acids⁸. This is a particular advantage since the lower dosage of lovastatin type HMG-CoA reductase inhibitor contributes to reduced side effects as well. ⁸ Heber D. et al. Cholesterol Lowering effects of Proprietary Chinese red yeast rice dietary supplement, Am J Clin Nutr 1999, 69:231-236.

How to Make the Invention

Materials-Red Yeast Rice Extract

Red Yeast Rice extract can be purchased as a nutritional supplement in the United States. Preferred sources include DRACO Natural Products (539 Parrott St., San Jose, Calif. 95112) Red Yeast Rice Extract 10:1, and the Thorne Research product, Choleast. Purchased Red Yeast Rice preferably should contain 0.2% or more of Monacolin K and 0.5% or more of Total Monacolins. We prefer to purchase the extract rather than manufacture it.

Red Yeast Rice Extract can also be manufactured directly from the yeast Monascus purpureus. The red yeast rice is the extract of fermenting-white rice, preferably non-glutinous white rice, with a Monascus purpureus strain of yeast, culturing said Monascus purpureus strain in a culture medium comprising rice at a temperature of about 15° C. to about 35° C. for a period of about 2 to about 20 days to provide a crude fermentation product containing red rice; drying said crude fermentation product to obtain red yeast rice, and then extracting said red yeast rice with a solvent-to provide an extract; and drying said extract to remove the solvent and produce red yeast rice extract. The solvent is preferably either aqueous ethanol or just water. Other culture media may be added to the rice in the culturing step. For example, sugar, an additional carbon source, and one or more items chosen from the group consisting of glycerine, malt, and potato juice; thick beefjuice, and defoamer.

The Red Yeast Rice extract is water soluble and is not soluble in the Omega-3 Oil. A dispersant is preferably used to keep the Red Yeast Rice extract in suspension. One preferred dispersant is 70% silica bamboo with Lysine made from Sunflower oil. A suitable mixture is to vigorously mix 24 weights of Red Yeast Rice Extract with 36 weights of Fish Oil, 1.2 weights of Bamboo, and 1.8 weights of Lysine. The mixture may then diluted to the desired omega oil to red yeast rice extract ratio. The omega-3/red yeast rice extract is then preferably encapsulated in soft gelatin capsules. Preferably the capsules are of such a size that an integral number of capsules comprise a daily dosage of the mixture.

A dosage of the mixture further preferably includes antioxidants an such as Rosemary, vitamin E, astaxanthine, carnitine, ascorbyl palmitate, tocopherols or other antioxidants known in the art for stabilizing fish oil.

It is preferred to use a high quality source of omega-3, which is rich in EPA and DHA, preferably at least 70% Omega-3 Oils, which Omega-3 Oils are preferably 70% EPA+DHA, and more preferably 85% or more EPA+DHA. A daily dose of Omega-3 oils is preferably 1-4 grams of Omega-3. One possible source is a balanced Omega-3 formula such as Nordic Naturals, Inc. ProOmega which comprises 70% Omega-3 Oils which Omega-3 Oils are 50.8% EPA, 35.1% DHA and 14.1% other Omega-3. Another preferred omega-3 source is Nordic Naturals Pro-EPA, whose Omega-3 comprise 69.1% EPA, 16.3% DHA, and 14.6% other omega-3. Still another preferred source is Nordic Naturals Pro-EFA xtra which comprises 56.9% EPA, 14.7% DHA, 17.2% GLA, and 11.2% other Omega-3 . The Pro-EFA xtra formula adds an Omega-6, GLA, and makes it a powerful anti-inflammatory mixture.

### EXAMPLES

One preferred embodiment is a daily dosage taken to reduce cholesterol and triglycerides comprising 1.2 grams per day of Red Yeast Rice Extract and 1700 mg of Nordic Naturals ®ProOmega® (which contains 70% Omega-3 Oils which are 85.9% EPA+DHA, so that 1700 mg will supply 1000 mg/day of EPA=DHA). Here the ratio of fish oil to RYR is 1700/1200=1.4. To make the mixture add 1200 weights of Red Yeast Rice Extract to 1700 weights of fish oil. Add 58 weights (2%) of 70% silica bamboo and 87 weights (3%) of Lysine and mix thoroughly to make a stable suspension. Fill gelatin capsules with the mixture such that an integral number of capsules dispenses 3045 grams of mixture.

Another embodiment is a daily dose comprising 1.2 grams per day of Red Yeast Rice extract and 3400 mg of fish oil such as ProOmega®. Here the ratio of fish oil to Red Yeast Rice is 2.8 and will supply 2000 mg/day of EPA+DHA.

Still another embodiment is a daily dose comprising 2.4 grams per day of Red Yeast Rice extract and 3400 mg of fish oil. Here the ratio of fish oil to Red Yeast Rice extract is 1.4.

Still another embodiment is a daily dose comprising 2.4 grams per day and 6800 mg of fish oil. Here the ratio of fish oil to Red Yeast Rice is 2.8.

Although the present invention has been described in considerable detail with reference to certain preferred versions thereof, other versions are possible. Therefore the spirit and scope of the appended claims should not be limited to the preferred versions herein.

What is claimed is:

1. A medicament comprising red yeast rice extract dispersed in fish oil

2. The medicament of claim 1, wherein the fish oil comprises at least 60% of Omega-3 oils.

3. The medicament of claim 2, wherein said red yeast rice extract comprises at least 0.1% monacolin K.

4. The medicament of claim 2, wherein said red yeast rice extract comprises at least 0.2% monacolin K and at least 0.4% total monacolins.

5. The medicament of claim 2, wherein the fish oil comprises 70% of Omega-3 oils.

6. The medicament of claim 5, wherein the Omega-3 oils comprise 50% EPA and 35% DHA.

7. The medicament of claim 5, wherein the Omega-3 oils comprise 69% EPA and 16% DHA.

8. The medicament of claim 5, wherein the weight ratio of fish oil to red yeast rice extract is in the range between about 1.4 to 2.8.

9. The medicament of claim 8, further comprising a dispersing agent.

10. The medicament of claim 9, wherein the dispersant comprises 3% Lysine and 2% Bamboo.

11. The medicament of claim 9, further comprising a soft gelatin capsule into which the fish oil, red yeast rice extract and dispersing agent are loaded.

12. The medicament of claim 11, wherein a daily dose of the medicament is delivered by an integral number of capsules.

13. The medicament of claim 12, wherein the daily dose of medicament comprises about 1000 mg. of EPA plus DHA and 1200 mg of Red Yeast Rice extract.

14. The medicament of claim 12, wherein the daily dose of medicament comprises about 2000 mg. of EPA plus DHA and 1200 mg of Red Yeast Rice extract.

15. The medicament of claim 12, wherein the daily dose of medicament comprises about 2000 mg. of EPA plus DHA and 2400 mg of Red Yeast Rice extract.

16. The medicament of claim 12, wherein the daily dose of medicament comprises about 4000 mg. of EPA plus DHA and 2400 mg of Red Yeast Rice extract.

17. The medicament of claim 11, further comprising an antioxidant.

18. The medicament of claim 11, wherein the antioxidant is chosen from the group consisting of Rosemary, vitamin E, astaxanthine, carnitine, ascorbyl palmitate, tocopherols.

19. A method of reducing serum cholesterol and triglycerides in humans comprising administering a daily dosage comprising EPA, DHA, and Red Yeast Rice extract delivered in an integral number of gelatin capsules.

20. The method of claim 19, wherein a daily dosage comprises about 4000 mg. of EPA plus DHA and 2400 mg. of Red Yeast Rice extract.

21. The method of claim 19, wherein a daily dosage comprises about 1000 mg. of EPA plus DHA and 1200 mg. of Red Yeast Rice extract.

22. The method of claim 19, wherein a daily dosage comprises about 2000 mg. of EPA plus DHA and 1200 mg. of Red Yeast Rice extract.

23. The method of claim 19, wherein a daily dosage comprises about 2000 mg. of EPA plus DHA and 2400 mg. of Red Yeast Rice extract.
